(19) United States
(12) Patent Application Publication
(10) Pub. No.: US 2014/0155869 A1
(43) Pub. Date: Jun. 5, 2014

(54) SPATULATED CANNULA TIP FOR USE WITH ROTATIONAL POWER ASSISTED LIPOSUCTION EQUIPMENT

(71) Applicant: William Seare, Carlsbad, CA (US)

(72) Inventor: William Seare, Carlsbad, CA (US)

(21) Appl. No.: 13/874,457

(22) Filed: Apr. 30, 2013

Related U.S. Application Data
(60) Provisional application No. 61/640,673, filed on Apr. 30, 2012.

(51) Int. Cl. A61M 1/00

(52) U.S. Cl. CPC A61M 1/008
USPC 604/542

(57) ABSTRACT

Spatulated cannulae for use with negative pressure or liposuction equipment provide a myriad of benefits including reduced recovery time, improved procedural efficiency and others. A spatulated cannula generally comprises a planar spatula tip disposed at a distal end of a cannular body. The spatulated cannulae are particularly useful with power assisted liposuction (PAL) instruments, especially rotational poser assisted liposuction equipment.

### CROSS-REFERENCE TO RELATED APPLICATIONS

This application claims benefit of priority with U.S. Provisional Ser. No. 61/640,673, filed Apr. 30, 2012, and titled “SPATULATED CANNULA TIP FOR USE WITH ROTATIONAL POWER ASSISTED LIPOSUCTION EQUIPMENT”; the contents of which are hereby incorporated by reference.

### BACKGROUND OF THE INVENTION

1. Field of the Invention

This invention relates to cannulae for liposuction surgery; and more particularly to such cannulae adapted with a spatulated tip for improved separation of body materials and penetration through tissues.

2. Description of the Related Art

Liposuction was first introduced to the United States by Dr. Yves-Gérard Illouz's. As liposuction has become increasingly practiced, many different modalities to enhance the removal of fat have been introduced, including increasingly smaller cannulae, different size and shape of various openings or orifice patterns in the cannulae, expanded baskets at the orifices, and other irregularities on or adjacent to the orifices. The “power assisted lipo”, or “PAL”, was introduced by Microaire® and includes a cannula configured to translate forward and backward along the axis of penetration, the power assisted lipo mechanism being powered by an electric motor. The introduction of PAL greatly reduced the time of lipo surgeries and improved lipo procedures in terms of healing time and other collateral benefits. Recent refinements to the PAL system include cannulas flared near the ends in the section where the holes are positioned, and the so-called “basked-cannulas”, also were a way of trying to remove fat more efficiently. These aggressive cannulae have been associated with increased vessel damage and skin loss, but can be used carefully in indicated cases. However, most liposuction surgeons universally prefer rounded or bullet ends on their cannulas.

Almost exclusively, cannula tips are blunt and the cannula body is cylindrical. As the cannula is moved longitudinally through the tissues, fat and fibrous tissue are presented to the holes within the cannula and the negative pressure pulls the presented tissues partially into the opening(s) along the cannula and further axial movement then avulses (pulls the tissue from) the surrounding tissues, thereby detaching it from within the body and allowing it to be moved along the inside of the cannula and eventually into a collection canister, where it can be disposed of, or used for autologous fat grafting (AFG) and transplanting back into the body in places such as the face, breasts, or buttocks. Many blunt tip designs have been described in the art, but sharper tips generally have not caught on clinically, because of the dangers of penetrating vital structures, such as the chest, abdominal cavity, retroperitoneal kidney area or adjacent muscle masses. In rare patients, where the fat contains very little fibrous tissue, movement of the cannula into untouched fatty tissue to avulse the fat requires very little force and/or effort. However, the majority of patients have some degree of fibrous tissues within the fat, creating difficulties in the movement, and direction of the cannula through the tissues and in removing the fat or scar tissue. Revision lipo cases have much greater fibrous tissue and can be difficult or impossible to remove the scar tissue and fat with standard technologies.

Recently, a “Power X” technology was approved by the FDA, and became available to surgeons in the US in early May, 2011. This technology uses an electrically powered geared-motor within a hand piece, connected to a controller that allows the degrees of rotation and counter rotation and the frequency of rotations to be controlled. It is different from many past such rotational apparati for lipo, in that it rotates first through one rotational arc up to 720 degrees and then rotates in the other direction up to 720 degrees. This refinement ensures that tissue does not become wrapped around the cannula tip thereby damaging and fowling the tissues on the cannula. Power X is helpful for the maximum benefit of certain embodiments herein, however the spatula-tipped cannula can also be rotated by hand, but with much more effort and loss of efficiency.

### SUMMARY OF THE INVENTION

Spatulated cannulae are disclosed for use with power assisted liposuction equipment, the cannulae being useful for procedures such as infiltration of fluids, or extraction of cells and/or tissues within a body. The spatulated cannula tip has been found by the inventor to be especially beneficial for the infiltration of fluids for hydration, infiltration, hemostasis and/or anesthesia of tissues beneath the skin, and for use in the field of liposuction, liposculpture, lipo, suction assisted lipectomy (SAL) and otherwise the removal of fat and/or other tissues from within a body utilizing negative pressure (suction).

In accordance with embodiments herein, a spatulated cannula generally comprises an elongated cannular shaft having a proximal end configured to engage with a negative pressure liposuction device and a distal end configured for surgical insertion within a body. The spatulated cannula further comprises a planar tip disposed at the distal end of the cannular shaft. Additionally, the spatulated cannula generally comprises one or more apertures disposed along the cannular shaft in a pattern sufficient for communication of fluids and body materials in a liposuction procedure.

### BRIEF DESCRIPTION OF THE DRAWINGS

Features and benefits of the claimed invention will be further understood upon a review of the following detailed description and in particular when reviewed in conjunction with the appended drawings, wherein:

FIG. 1A illustrates a top view of a spatulated cannula in accordance with an embodiment.

FIG. 1B illustrates a side view of the spatulated cannula of FIG. 1A.

FIG. 2A illustrates a top view of a regular spatulated cannula tip in accordance with an embodiment.

FIG. 2B illustrates a top view of an elongated spatulated cannula tip having a spatula tip with an expanded length in accordance with an embodiment.

FIG. 2C illustrates a top view of a widened spatulated cannula tip having a spatula tip with an expanded width in accordance with an embodiment.

FIG. 2D illustrates a top view of a narrowed spatulated cannula tip having a spatula tip with a narrowed width in accordance with an embodiment.

FIG. 3A illustrates a top view of a spatulated cannula having a multi-portion spatula tip comprising a first planar spatula portion extending within a first plane, and a second planar spatula portion extending within a second plane orthogonal with respect to the first plane.

FIG. 3B illustrates a side view of the spatulated cannula of FIG. 3A.

FIG. 4A illustrates a top view of a spatulated cannula having a tapered spatula tip in accordance with an embodiment.

FIG. 4B illustrates a side view of the spatulated cannula of FIG. 4A.

FIG. 5A illustrates a side view of a spatulated cannula having a plurality of apertures being disposed about an elongated cannular shaft in a predetermined pattern optimized for communication of fluids and other materials during a liposuction procedure.

FIG. 5B illustrates a top view of the spatulated cannula of FIG. 5A.

### DETAILED DESCRIPTION OF THE PREFERRED EMBODIMENTS

In the following description, for purposes of explanation and not limitation, details and descriptions are set forth in order to provide a thorough understanding of the present invention. However, it will be apparent to those skilled in the art that the present invention may be practiced in other embodiments that depart from these details and descriptions without departing from the spirit and scope of the invention. Certain embodiments will be described below with reference to the drawings wherein illustrative features are denoted by reference numerals.

Spatulated cannulae are disclosed for use with power assisted liposuction equipment, the cannulae being useful for procedures such as infiltration of fluids, or extraction of cells and/or tissues within a body. The spatulated cannula tip has been found by the inventor to be especially beneficial for the infiltration of fluids for hydration, infiltration, hemostasis and/or anesthesia of tissues beneath the skin, and for use in the field of liposuction, liposculpture, lipo, suction assisted lipectomy (SAL) and otherwise the removal of fat and/or other tissues from within a body utilizing negative pressure (suction).

In accordance with embodiments herein, a spatulated cannula generally comprises an elongated cannular shaft having a proximal end configured to engage with a negative pressure liposuction device and a distal end configured for surgical insertion within a body. The spatulated cannula further comprises a planar tip disposed at the distal end of the cannular shaft. Additionally, the spatulated cannula generally comprises one or more apertures disposed along the cannular shaft in a pattern sufficient for communication of fluids and body materials in a liposuction procedure.

In accordance with various embodiments herein, many advantages of the disclosed spatulated cannulae include but are not limited to:

### Reduced Force of Penetration Through Fat and Scar Tissues

The force of introduction and movement through untouched tissues are greatly reduced with rotational movement and a spatulated tip. The force required with the Power X set at a frequency 420 rpm, with 360 degrees of rotation is amazingly decreased when moving through similar fibro-fatty tissue than with a manual bullet-tip cannula. With constant pressure of 25% of the force required to move the rotating bullet-tipped cannula, the spatula-tip cannula can be felt to work its way a few millimeters or centimeters at a time, gently through the tissues rather than releasing unexpectedly through fibrous tissues. This unintended advancement through tissues comes with risks of penetration unwanted tissues, with the potential of unintended injury. Many times, the greater the density of the tissue being penetrated, the greater the percentage of reduction of the force required to safely penetrate the fibro-fatty tissues. With the PAL and its longitudinal jigsaw-like advancements, the force is also reduced, but not as much as with the oscillating rotations of the Power X.

### Finding the Plane of Least Resistance

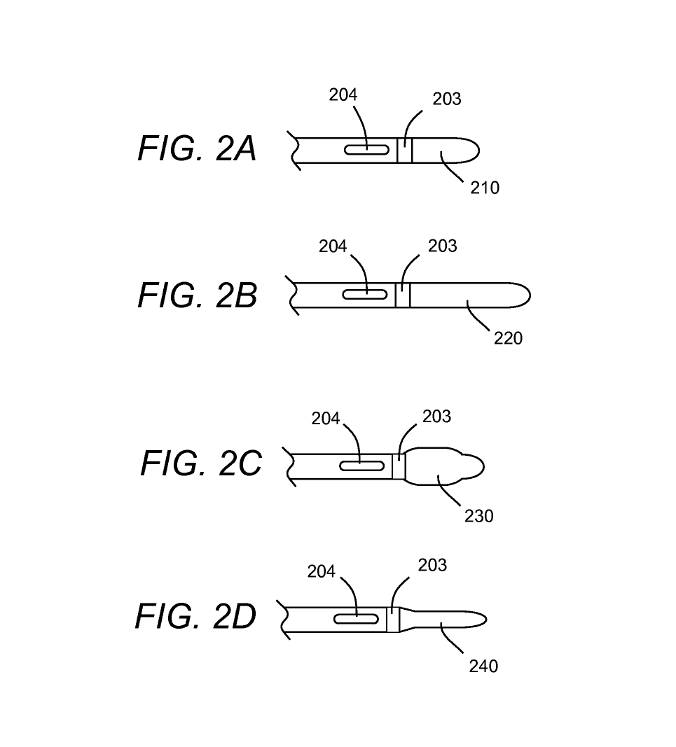

Another extension of this reduction of force, is finding the plane of least resistance. Collagen is lamellar in construction and even scar tissue has these lamellae. With the Power X rotations, with constant reduced pressure, the spatula tip will penetrate with the blade in parallel and then finishing the rotations, will open up the lamella, stretching and creation a hole with looser tissue for the next penetration. One can also rotate manually the handle through 180 degrees and allow the spatula-tip find the plane of least resistance, but the opening effect is much less. For the Power X, the reciprocating rotational motion of the cannula allows the flattened spatula-tip to find a plane or orientation of least resistance through the fibro-fatty tissues, creating a rotation-find-release and repeated a rotation-find-release motion through the tissues, gently opening them up while moving rhythmically through the tissues. This force can be 25%-50% of a non-spatulated tip and sometimes even more. With PAL and just the spatula tip without rotation, there may be 10-15% reduction. With the Pal with manual rotation it can be 15%-25% or slightly more. This appears to be a different mechanism than the rotation effect. It appears to be somewhat like a jack-hammer, where repeatedly pocking an area with less force, gradually opens it up as opposed to a greater large force. By manual rotation with the PAL, it appears to find the fiber orientation that has the least resistance. This also moves through the tissue in a rotation-find-release the areas of least resistance, but it is not as efficient as the rotation effect. By making the tip more tapered and narrower at entrance, as in FIG. 2D, this force can even be reduced more, but with an increased risk of penetrating unwanted tissues and should only be used by experts, but has increased advantages.

### Decreases the Introduction Force

Another advantage of the invention that also decreases the introduction force is the beneficial opening and spreading of the fibro-fatty tissues caused when the flat spatula tip, finding the plane of least resistance, penetrates the tissues up to the length of the spatula-tip and rotates about 90%, expanding the thickness of the spatula tip to the width of the tip, generally the same width as the diameter of the cannula, thereby creating an opening the size of the cannula, allowing an easier introduction. After the stretching and opening of the tissue, the cannula openings toward the handle section can then pull the fibro-fatty tissues into the openings or “apertures” and remove the tissue. When this is done multiple times, even very dense fibro-fatty tissue can be treated, such as found in patients who have had previous lipo surgeries. Here the tissues can only be penetrated with increased force and difficulty by the surgeon and risking penetration of vital tissues. The rotation and opening effect after multiple repetitions actually softens and makes removals that are impossible otherwise, and makes it possible with less energy by the surgeon and risk to the patient.

### Improved Guidance of the Cannula

Another advantage is the ability to better guide the cannula through tissues. The cannula tip is at the end of a very long cannula section, and even when this may be somewhat shorter with shorter cannulae, the lever arm to control the cannula tip is quite long. Many times the cannula tip finds the tunnel through the tissue that already has the fibro-fatty tissue removed, repeatedly going back into this tunnel, resulting in less and less tissue being removed. It also tends to create over suctioned areas, and under suctioned areas with fibro-fatty tissue in areas that have never been penetrated by a cannula. The spatula-tip, along with the rotational effect of the Power X creates a guide-ability of the tip that was entirely unexpected. Much like the elevators of a canard airplane, where the “tail” of the airplane is in front, when gentle pressure toward the direction the surgeon wishes to move the cannula tip is applied, when the rotation of the spatula-tip's horizontal section is perpendicular to the direction of force, the spatula-tip tends to dive or move in this in this direction. The opening and removal of the tissue also help amplify this effect by softening the general area. These combined factors produce better guidance of the cannula tip, penetrating fibro-fatty globules without having the fat bounce off and dodge to the side.

### Fragmentation

A major unexpected advantage of the spatula-tip when used with reciprocating Power X system is the actual fragmentation of fat globules and fat, including partial and/or complete disruption of the fat from the area surrounding vessels, nerves and collagen scaffolding as the spatula tip as it is gently being pushed and withdrawn through the tissues. This process is more efficient if the cannula is pushed slowly, and then the suction holes further proximally on the cannula can more easily aspirate the fat that has been loosened or separated. This also assists significantly in reduction of suction time and the energy and number of penetrations of a particular area required reducing the fat. This reduction in the number of times of penetration also causes fewer traumas to the vessels supplying the overlying skin so more fat can be removed more safely and completely without skin loss from vessel damage. Part of the explanation for the reduction in damage of vascular structures is that instead of the fat actually being suctioned partially into the holes and then avulsed with the very sharp edge of the walls of the thin-wall stainless cannula, the fat is pre-loosened so that much less force and cutting is needed to get the fat moving down the cannula.

### Penetrated Moveable or Harder to Penetrate Sections

Another effect of the rotation spatula-tip cannula is more direct penetration of globules of fibro-fatty tissue. In observing the suctioned tissues after the Power X spatula-tip cannula in mini-abdominoplasties, where the superficial layer of skin is removed, there are many less skipped areas, or areas that have not been penetrated by the cannula. This effect can be directly observed in these same dissections, where when trying to penetrate the fibro-fatty globules, some of which are about 2 cm in diameter, the bullet-tip cannulas push these to the side and tend to penetrate already suctioned areas. With the spatula-tip cannula, without any manual or rotatory motion, this effect is about the same. However, with manual or Power X rotation, the spatula-tip cannula can be seen to “drill” into these globules and dislodge and aspirate the contained fat.

Vibratory Effects with Reduction of Pain

There is an interesting phenomenon that happens with the rotatory oscillations in relation to the perceived pain, especially during the early stages of the tumescent anesthesia where the local anesthetic, usually Lidocaine, numbs the C pain fibers. Some of the larger bundles probably take longer to anesthetize, so there is still the ability to sense stretching and pain. Also, when using it for the infusion cannula for the tumescent anesthesia, there is no anesthesia present. The inventor has found that frequency that occurs with the Power X at the upper settings of the frequency settings 340-420/min seem to confuse the pain fibers or overwhelm the vibration fibers so that there is much less perceived pain. Another mechanism appears to be that the oscillation does not allow the larger pain fibers or nerve bundles to be caught on top of the rounded end of the stretched nerve fiber as the cannula is advanced. Rather, the oscillations allow the end-on nerve stretching from the cannula to be shifted off to the side, causing much less stretch. At the available Power X settings, the vibratory advancements of the cannula are much less stimulating to pain receptors than the blunt stretching that happens with a rounded cannula. These mechanisms are not just because of the decreased cross-sectional area of the tip in the spatula-tip compared to a bullet-tip, because this effect essentially goes away when the powered oscillations are stopped. This effect is also enhanced somewhat in fibrous tissue, presumably because of the increased force required to advance the cannula, tending to stretch and compress the nerves less at the point of contact with the tip.

### Infusion of Fluids

Similar advantages of the spatula-tip are found when using the power rotation of the cannula for infusion of fluids within the body, such as for anesthesia and specifically tumescent anesthesia for liposuction surgery. Major advantages include:

(1) GUIDABILITY—in general, infusion cannulae are much thinner than the suction cannulae and guiding them is more of a problem than with suction cannulas because of their bending. The guidability is even more important with these surgical instruments. Because of their smaller diameter, it would be much easier to penetrate unwanted structures, including the lungs and abdomen; and

(2) SAFETY—much lower forces and gentle continuous pressure allows the cannulae to work through the sometimes very dense and fibrous tissues without major jerks or unwanted long excursions, such that the limits of the fibrous tissue are reached and soft tissues are entered.

Lubricity Created with the Spatulated Cannula

An apparent unexpected advantage of the spatula-tip cannula is that as the fat is broken-up or macerated, more of the oil from within the cells that may be lysed coats the cannula making it more slippery going through the tissues. With a single stroke of the cannula and then testing the slipperiness with a clean latex glove there is more of a lubrication layer along the cannula compared to a bullet-tip cannula in view of the same test. This lubrication does not convert to more free oil within the aspirate, because if one performs one area with the bullet-tip cannula and the other with the spatula-tip cannula, there is less free oil when the aspirate is left to settle for 2 hours. Also, there is less blood concentration in the aqueous phase of the aspirate with the spatula-tip cannula.

### Better Fat Quality for Autologous Fat Grafting (AFG)

From the lubricity discussion, it can be seen that the actual aspirate harvested for AFG from the spatula-tip cannula is of better quality than for the bullet-tip cannula because there is less blood within the aspirate as well as less free oil which means that the decanted fat (aqueous layer removed after being allowed to settle) many times doesn't need to be washed with normal saline. This washing potentially can harm both the fat aggregates and the stem cells present within the aspirate.

### EXAMPLES

Various embodiments are described wherein a spatulated cannula comprises an elongated cannular body having a hollowed inner body portion. The cannular body comprises a proximal end and a distal end. The distal end is configured to couple with a power assisted liposuction (PAL) equipment, especially a rotational PAL such as the Power X liposuction device and others. The proximal end of the cannular body is configured with a planar spatulated tip. One or more apertures may be disposed about the cannular body adjacent to the tip in a pattern configured for the communication of fluids and other body materials through the cannula.

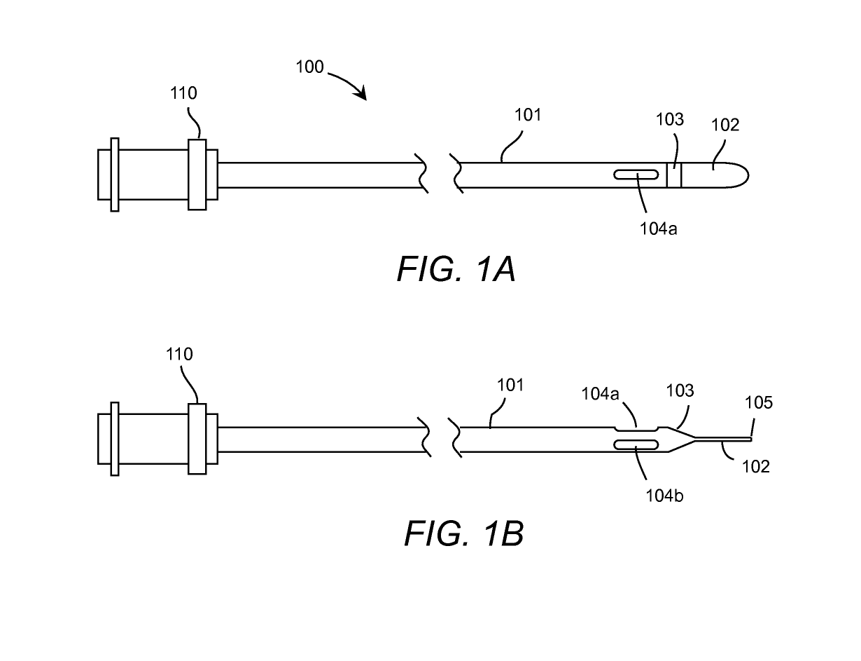

Turning now to the drawings, FIGS. 1A & 1B illustrate a top view and side view, respectively, of a spatulated cannula 100 in accordance with an embodiment. The spatulated cannula comprises a cannular body 101 extending from a proximal end to a distal end. A coupler 110 is disposed at the proximal end and configured to engage with a power assisted liposuction device. A plurality of apertures 104a; 104b are circumferentially disposed about the cannular body near a distal end thereof and adjacent to a distal tip 105. A third aperture is not shown, however the three apertures are each disposed about 120 degrees around the circumference of the cannular body, forming what may be referred to as a “Mercedes” pattern. The cannula further comprises a gradual taper portion 103 extending from the cannular body to a planar spatulated tip 102 at the distal end.

FIGS. 2(A-D) illustrate various embodiments of a spatulated cannula tip.

FIG. 2A illustrates a top view of a spatulated cannula tip in accordance with an embodiment. An aperture 204 is shown being disposed adjacent to the spatulated cannula tip at the distal end of the cannula. A tapered portion 203 extends from the cannular body to the spatulated tip. The tapered portion gradually varies the thickness of the tip going from the spatula tip to the cannular body. The spatula tip 210 comprises a planar elongated portion having a rounded point.

FIG. 2B illustrates a top view of a spatulated cannula tip having a spatula tip with an expanded length in accordance with an embodiment. An aperture 204 is shown being disposed adjacent to the spatulated cannula tip at the distal end of the cannula. A tapered portion 203 extends from the cannular body to the spatulated tip. The tapered portion gradually varies the thickness of the tip going from the spatula tip to the cannular body. The elongated spatula tip 220 comprises a planar elongated portion having a rounded point. Here, the planar elongated portion extends longer than the spatula tip 201 of FIG. 2A.

FIG. 2C illustrates a top view of a spatulated cannula tip having a spatula tip with an expanded width in accordance with an embodiment. An aperture 204 is shown being disposed adjacent to the spatulated cannula tip at the distal end of the cannula. A tapered portion 203 extends from the cannular body to the spatulated tip. The tapered portion gradually varies the thickness of the tip going from the spatula tip to the cannular body. The widened spatula tip 230 comprises a planar elongated portion having a rounded point. Here, the planar elongated portion extends wider than the spatula tip 201 of FIG. 2A. Additionally, the widened spatula tip 230 comprises a width greater than a diameter of the cannular body.

FIG. 2D illustrates a top view of a spatulated cannula tip having a spatula tip with a narrowed width in accordance with an embodiment. An aperture 204 is shown being disposed adjacent to the spatulated cannula tip at the distal end of the cannula. A tapered portion 203 extends from the cannular body to the spatulated tip. The tapered portion gradually varies the thickness of the tip going from the spatula tip to the cannular body. The narrowed spatula tip 240 comprises a planar elongated portion having a rounded point. Here, the planar elongated portion extends narrower than the spatula tip 201 of FIG. 2A. Additionally, the narrowed spatula tip 240 comprises a spatula width less than a diameter of the cannular body.

Each of the above embodiments of FIGS. 2(A-D) serves a distinct function during a liposuction procedure. For example, as the tip is narrowed, the tip becomes more susceptible to puncture and thus is only used where necessary to separate fibrous tissue away from vital organs. Accordingly, it may be beneficial to provide a kit or set of spatulated cannulae for interchangeable use during a procedure.

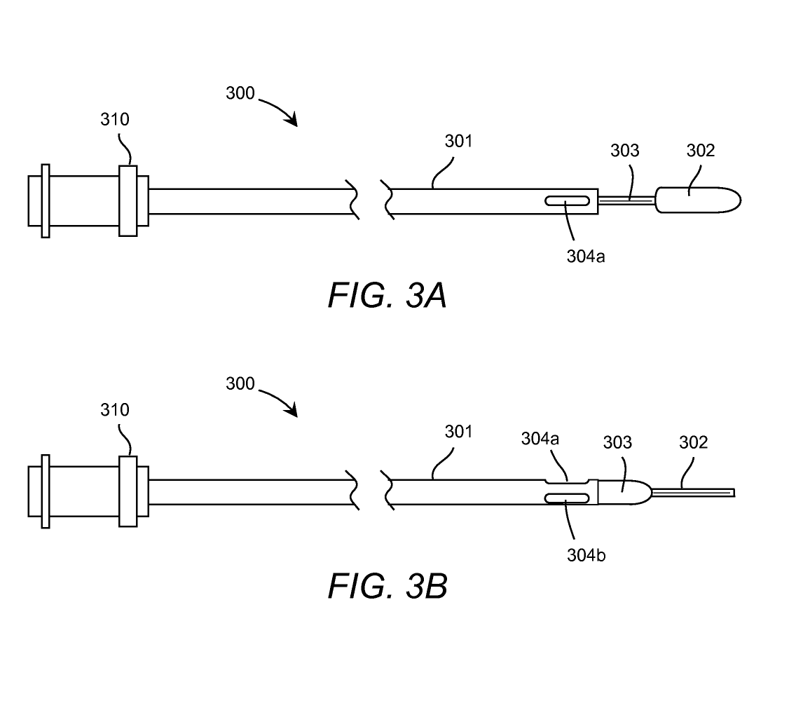

In another embodiment, FIGS. 3A & 3B illustrate a top view and side view, respectively, of a spatulated cannula 300 having a multi-portion spatula tip comprising a first planar spatula portion 302 extending within a first plane, and a second planar spatula portion 303 extending within a second plane orthogonal with respect to the first plane. The spatulated cannula comprises a body portion 301 having a proximal end configured with a coupler 310 for coupling with a power assisted liposuction device, and a distal end comprising the multi-portion spatula tip. One or more apertures 304a; 304b can be disposed about the surface of the cannular body adjacent to the spatulated tip and configured in a pattern for communicating fluids and body materials through the lumen space of the cannula.

In the illustrated embodiment of FIGS. 3(A-B), the first planar spatula portion is disposed ninety degrees with respect to the second spatula, or in orthogonal relation. However, in certain other embodiments the first and second planar spatula portions may be oriented at any angle. Furthermore, other conceived embodiments may include three or more planar spatula portions being consecutively aligned along a distal tip of the cannula and oriented at one of a myriad of possible configurations with respect to angles or alignments thereof. Thus, FIG. 3 merely illustrates an example of a multi-portion spatulated cannula having two or more spatulated portions extending to form a distal tip; those having skill in the art will be able to recognize a myriad of possible configurations having a plurality of spatula portions.

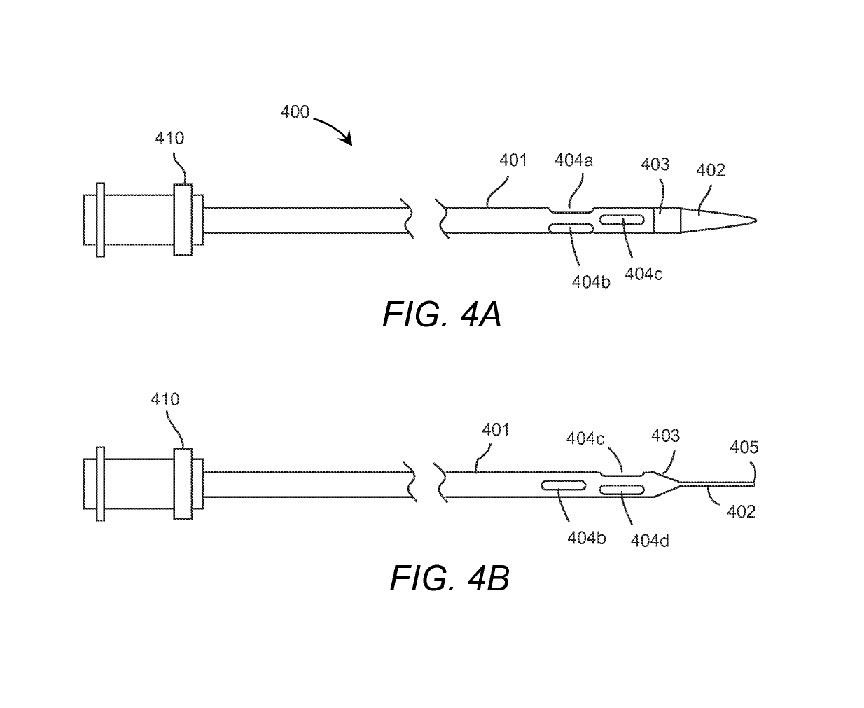

In another embodiment, FIGS. 4A&4B illustrate a top and side view, respectively, of a spatulated cannula 400 having a tapered spatula tip 402 in accordance with an embodiment. The spatulated cannula comprises a body 401 extending from a proximal end to a distal end. At the proximal end resides a coupler 410 for coupling the cannula to a liposuction device. A plurality of apertures 404a; 404b; and 404c are disposed along a surface of the body 401 in a pattern configured for the communication of body fluids and materials through the lumen cavity inside the cannular body portion and into the liposuction device. A tapered portion 403 extends from the cannular body at the distal end to the planar spatulated tip 402. The planar spatulated tip 402 comprises a planar volume extending toward a distal tip 405.

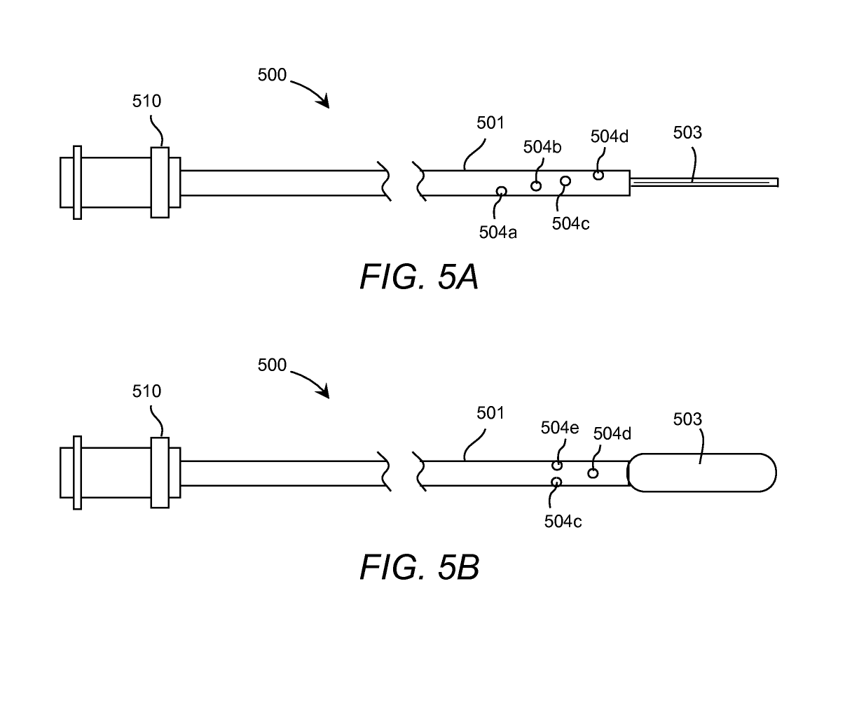

In yet another embodiment, FIGS. 5A&5B illustrate a side and top view, respectively, of a spatulated cannula 500 having a plurality of apertures 504(a-e) being disposed about an elongated cannular shaft in a predetermined pattern optimized for communication of fluids and other materials during a liposuction procedure. The spatulated cannula 500 comprises a body portion 501 extending from a proximal end to a distal end. A coupler 510 is disposed at the proximal end and configured to couple with a liposuction device. The apertures 504(a-e) are disposed in a Christmas-tree pattern along the cannular body and adjacent to the distal end thereof. A spatulated tip extends outwardly from the cannular body. In this example, no tapered portion extends between the cannular body and the spatulated tip; however such a tapered portion may be incorporated in similar embodiments as indicated above.

In various embodiments, the coupler can be an adapter for engaging a Power X suction and rotation liposuction device.

In another aspect, a method for using a handheld liposuction device comprising rotational power assisted motion, comprises: (i) combining a spatulated cannula with the liposuction device; and (ii) performing a liposuction procedure.

In an embodiment of the above method, the spatulated tip comprises a multi-portion spatulated tip having a first spatula portion and a second spatula portion, the first spatula portion having a first planar volume being disposed in a first plane and the second spatula portion having a second planar volume being disposed in a second plane, wherein the first plane is distinct from the second plane.

Other features and benefits of the various embodiments will be recognized by those having skill in the art upon a thorough reading of the disclosure, and in view of the claims.

I claim:

1. In a handheld liposuction device having a single cannula, a means for holding and maneuvering the cannula, and a means for driving the cannula in a rotating motion, the improvement comprising:
a planar spatulated tip extending outwardly from a body of the cannula.

2. The improvement of claim 1, wherein said spatulated tip comprises a rounded point.

3. The improvement of claim 1, wherein said spatulated tip comprises a width greater than a diameter of the cannular body.

4. The improvement of claim 1, wherein said spatulated tip comprises a width less than a diameter of the cannular body.

5. The improvement of claim 1, comprising a tapered volume extending between the cannular body and the spatulated tip.

6. The improvement of claim 1, wherein said spatulated tip comprises a first spatula portion and a second spatula portion, the first spatula portion having a first planar volume being disposed in a first plane and the second spatula portion having a second planar volume being disposed in a second plane.

7. The improvement of claim 6, wherein the first plane is distinct form the second plane.

8. The improvement of claim 6, wherein the first plane is orthogonal with the second plane.

9. The improvement of claim 6, comprising three or more spatula portions.

10. The improvement of claim 1, wherein the spatulated tip comprises tapered sides leading to a rounded point.

11. The improvement of claim 1 having a coupler disposed at a proximal end of the cannular body, the coupler being configured to engage with a rotational power assisted liposuction device.

12. The improvement of claim 1, comprising one or more apertures disposed about a surface of the cannular body.

13. The improvement of claim 12, wherein said apertures are configured in a pre-determined pattern for optimizing communication of fluids and body materials through an inner cavity of the cannula.

14. The improvement of claim 13, wherein said pre-determined pattern is one of: a Mercedes pattern, or a Christmas tree pattern.

15. A spatulated cannula for use with a liposuction device, comprising:
a cannular body extending from a proximal end to a distal end;
a coupler disposed at the proximal end and configured to engage with a power assisted liposuction device;
one or more apertures disposed about a surface of the cannular body; and
a planar spatula tip extending outwardly from the cannular body at the distal end thereof;
the planar spatula tip being one of: a regular spatula tip; a widened spatula tip; a narrowed spatula tip; an elongated spatula tip; or a multi-portion spatula tip.

16. The spatulated cannula of claim 15, wherein said spatulated tip comprises a first spatula portion and a second spatula portion, the first spatula portion having a first planar volume being disposed in a first plane and the second spatula portion having a second planar volume being disposed in a second plane, wherein the first plane is distinct from the second plane.

17. A method for using a handheld liposuction device comprising rotational power assisted motion, comprising:
combining a spatulated cannula with the liposuction device; and
performing a liposuction procedure.

18. The method of claim 17, wherein said spatulated tip comprises a first spatula portion and a second spatula portion, the first spatula portion having a first planar volume being disposed in a first plane and the second spatula portion having a second planar volume being disposed in a second plane, wherein the first plane is distinct from the second plane.
